(19) United States
(12) Patent Application Publication
(10) Pub. No.: US 2004/0092904 A1
(43) Pub. Date: May 13, 2004

(54) Diaper

(76) Inventors: Carlos De Silva Macedo JR., Sao Jose Dos Campos (BR); Eduardo Cezar Andreo Aledo, Sao Jose Dos Campos (BR); Altair De Paula Vitor, Sao Jose Dos Campos (BR); Francisco Javier Valdivia Hernandez, Sao Jose Dos Campos (BR)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/399,288

(22) PCT Filed: Oct. 17, 2001
(86) PCT No.: PCT/BR01/00128

(30) Foreign Application Priority Data
Oct. 19, 2000 (BR) PI 0004936-0

(51) Int. Cl. A61F 13/15; A61F 13/20

(52) U.S. Cl. 604/396; 604/358; 604/385.27

(57) ABSTRACT

The present disclosure refers to a diaper comprising an absorbent core disposed within the diaper and provided with a continuous and ring shaped element in the groin region of the diaper and cooperating with a distal end of the diaper so as to close the diaper around the user's body.

### FIELD OF THE INVENTION

The present invention refers to a diaper for newborns, infants and geriatric cases, allowing immediate placement and sustained retention after successively opening and closing the diaper.

### BACKGROUND OF THE INVENTION

Generically, diapers are disposable articles of a multi-layered kind, having at least a body fluids permeable material layer in the inner side or the side in contact with the user's body and a body fluids impermeable material layer in the reverse or outer side of the diaper, with a generically flat and elongated core of an absorbent material disposed between said layers.

The body fluid permeable material layer is generally made of soft and non-irritating material. According to the present state of the art, it may be a sheet of woven or non-woven made form natural fibers (for example, wood or cotton fibers) or artificial fibers (for example, polyester or polypropylene), combinations of such fibers, a perforated plastic film, a porous or cross-linked foam and the like, Said layer can be of known hydrofilicity, showing a tendency to remain dry and can be a mono or multilayer material.

The body fluid impermeable material layer has the function of preventing the absorbed and retained fluid in the absorbent core form passing to the user's clothes or skin, it generally being a thin polyethylene film. However, said layer being impermeable to liquids, it may be permeable to vapors as well and, in such case, will be provided with small pores or made of a material which provides impermeability only to liquids.

The absorbent core of known disposable absorbent articles is usually formed of cellulose fibers, often comprising super-absorbent polymers capable of forming a gel in contact with liquids as well. Other absorbent materials, such as papers, peat moss, artificial fibers, foam and the like are also known.

There are many shapes and several sizes for state-of-the-art diapers, presenting, however, as a common trait the fact of being of the open kind with a pair of closing tabs or ends in the waistband, where, for placement in the user, the diaper is placed in an open position and afterwards its ends are joined, the diaper then being attached to the user's body.

In attempting to facilitate and assure the attachment of the diaper, especially in the case of newborns and infants, as well as to keep them safer against body fluids leakage, a diaper having an elastic ring was developed, which means it has no opening except for those provided for the user's for legs and trunk, they so-called pull-up diaper. Although the objectives regarding the prevention of leakage are achieved, the placement of such diaper in a user was inconvenient, since the infant's legs had to go through the orifices, this being a difficult task. Another inconvenience is the fact that such diaper construction does not allow adapting to very different physical constitutions, the range of use for each diaper size being small, which limits its use.

### SUMMARY OF THE INVENTION

It is an object of this invention to provide a pull-up type diaper that is capable of being opened without requiring removal of the diaper.

In accordance with the present invention there has been provided a diaper comprising a body fluid permeable material layer adapted to contact a user's body in use, a body fluid impermeable material layer, an absorbent core disposed between and enclosed by said respective fluid permeable and impermeable material layers, the diaper further including a pair of opposite side margins, a rear margin and a distal end, the distal end being opposite the rear margin and defining therebetween a longitudinal length, the rear margin being adjacent to both said opposite side margins, the rear margin and the distal end each having a respective transverse width, the rear margin comprising a continuous and ring shaped element adapted to be worn around a waist region of the user's body in use, wherein the distal end is freely extending and moveable with respect to the rear margin and being adapted to be releaseably attached to at least a portion of the rear margin.

### BRIEF DESCRIPTION OF THE FIGURES

The present invention will be described in more details in the following, based on one of the possible embodiments of the present invention, as shown in the following figures:

FIG. 1 is an upper plan view of an inner side of the diaper;

FIG. 2 is a perspective view of the diaper in a placement and retention position on the waist of a user not shown;

FIG. 3 is a perspective view of the diaper of FIG. 2 with a closing means;

FIG. 4 is a perspective view of the diaper showing another closing means.

### DETAILED DESCRIPTION OF THE FIGURES

The present invention is directed to a diaper comprising a body fluid permeable material layer adapted to contact a user's body in use, a body fluid impermeable material layer, an absorbent core disposed between and enclosed by said respective fluid permeable and impermeable material layers. The diaper has a pair of opposite side margins, a rear margin and a distal end. The distal end is opposite the rear margin and defines therebetween a longitudinal length. The rear margin is adjacent to both of the opposite side margins. The rear margin and the distal end each have a respective transverse width. The rear margin comprises a continuous, ring shaped element that is adapted to be worn around the user's waist in use. The distal end is freely extending and moveable with respect to the rear margin and is adapted to be releaseably attached to at least a portion of the rear margin.

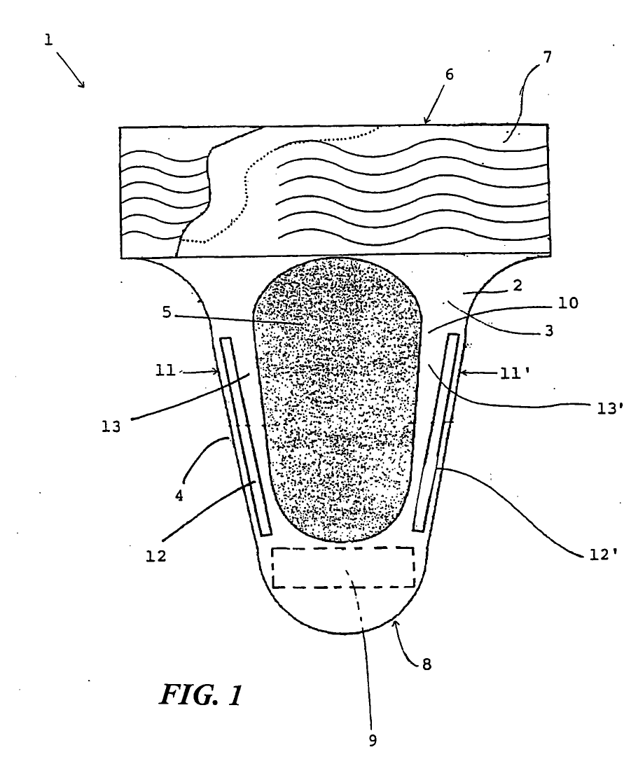

Referring now to FIG. 1, it is shown a diaper 1, comprising a body fluid permeable material layer 3 for contacting with the user's body and, in the reverse side and turned outwards, a body fluid impermeable material layer 4. Between said body fluid permeable material layer 3 and body fluid impermeable material layer 4 there is disposed an absorbent core 5. The absorbent core has a geometrical shape, that when viewed in a plan view, is shaped substantially similar to a truncated cone section having the base adjacent the rear margin.

The diaper has a rear margin 6 that is in the form of a continuous, ring shaped element 7, forming the diaper's waistband and which, is use, is adapted to be worn around a user's waist. Opposite to the rear margin 6 is a distal end 8, said distal end 8 being freely extending and moveable with respect to the rear margin 6. The distal end 8 preferably has a rounded border. The rear margin 6 and the opposite distal end 8 define therebetween a longitudinal length of the diaper 1.

The rear margin 6 and the distal end 8 each have respective transverse widths. The diaper of the present invention has a ratio of the transverse width of the rear margin to the transverse width of the distal, when measured in a plan view, of greater than 2, and is preferably about 3. As used herein, the terminology “in a plan view” refers to measuring the diaper when it is in a completely flattened or planar position, such that any elastic elements that would otherwise prevent a planar position are stretched in an amount sufficient to permit a planar orientation. However, elastic elements that are present in the diaper but are not in a location that would prevent a planar position are measured in a non-stretched condition. For example, if the rear margin includes elastic elements that allow the continuous, ring shaped element 7 to comfortably adapt to a user's waist, but do not otherwise prevent the diaper from being in a planar position, the width of the rear margin would be measured in a non-stretched condition. The ratio of the longitudinal length of the diaper 1 and the transverse width of the distal end 8 varies between 0.6 and 1.3, and is preferably about 1.0.

The distal end 8 is provided with an attachment element 9 that is adapted to attach the distal end 8 to a portion of the ring shaped element 7. The ring shaped element 7 is preferably formed by two superposed portions that are joined together by transverse joining lines. The ring shaped element 7 may be formed joined by sealing, sewing or other processes for forming said ring shaped element 7. In a most preferred embodiment, the ring shaped element is provided with one or more elastic elements that are adapted to allow the ring shaped element 7 to expand or contract around a user's waist in use. The elastic elements may extend across the entire transverse width of the rear margin 6, or alternatively may be located in one or more discrete locations in the ring shaped element 7.

The diaper further includes an intermediate region 10 located between rear margin 6 and the distal end 8. In this intermediate region 10 there is disposed a relatively thin absorbent core 5, in order to provide a comfortable use. In a preferred embodiment, the absorbent core 5 has a caliper less than 5 mm, more preferably less than 4 mm and most preferably between 2.5 to 4 mm. The intermediate region 10 has a pair of opposite side margins 11, 11′, adjacently opposed to said rear margin 6 and said distal end 8. The side margins preferably converge from the rear margin 6 towards the distal end 8. Said side margins 11, 11′ are provided along a region thereof with respective elastic 12, 12′ for adjusting the diaper to the groin region of the user, thus preventing body fluids leakage in use. The elastic elements 12, 12′ are strip shaped and are adjacent to and parallel to said side margins 11 and 11′ having a length substantially equivalent to the length of the side margins 11, 11′ to which they are attached. The elastic elements 12, 12′ are preferably tensioned prior to placing them in the side margins 11, 11′. The elastic elements 12, 12′ are located close to each longitudinal side margin 11, 11′ and adjacent to the absorbent core 5, and when tensioned form wall regions 13, 13′ which are located along each side margin 11, 11′ thus providing two elastic corrugated walls. The function of such wall regions 13, 13′ is to prevent side leakage aided by elastics 12, 12′ shown as rectangular shaped stripes and parallel to the longitudinal side margins 11,

In one embodiment of the invention, the side margins 11, 11′ of the intermediate region 10 converge mutually and symmetrically in relation to the longitudinal axis from said rear margin 6 to the distal end 8, defining a substantially straight path and a constant slope angle, as measured between said respective side margin and the longitudinal symmetry axis of the diaper. The slope angle is between 5° and 75°, and is preferably 20°. In accordance with this embodiment, the diaper 1 has a geometrical shape similar to a transverse area of a truncated cone.

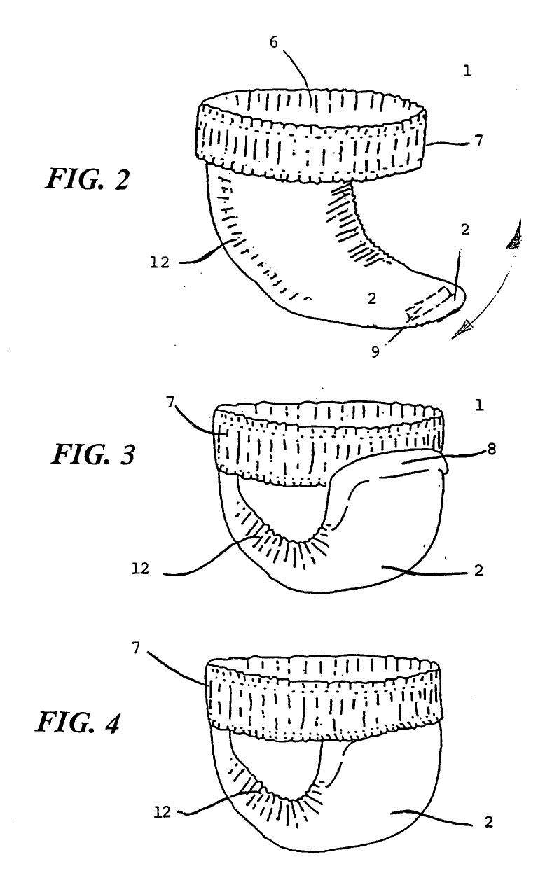

FIG. 2 shows the invention diaper during use. When diaper 1 is put on the user, it creates a radial elastic force component, compressing the user's trunk and allowing the correct attachment of diaper 1, aiding against leakage. The transverse width of the rear margin 6 forming the diaper's waist band is variable between respective minimum and maximum positions of ring shaped element 7 stretching, overcoming the size limitations of state-of-the-art diapers and attending a wider range.

Diaper 1 is kept on the user's waist by the closed waistband formed by ring shaped element 7, the diaper 2, freely oscillation along the rear margin 6 formed by ring shaped element 7, as indicated by the double arrow in the FIG. 2, for opening and closing without loosening the diaper form the user's waist. That is, the diaper is open in a vertical position and suspended from the waist. One of the advantages brought about by the present invention diaper is the autonomous toilet training for children during the diaper abandoning phase and during adaptation to underwear or panty, since the child opens the diaper retained in the waist.

FIG. 3 shows the diaper having a way of closing by means of the attachment element 9 retained in a portion of ring shaped element 7. The attachment element 9 may be placed in any of the layers 3 or 4 of the diaper 1, this placement variation being only a matter of desirability or production easiness. For example, FIG. 3 represents the diaper wearing during abandoning phase, whereas attached between the abdomen and a portion or ring shaped element 7, thus making it more difficult for the diaper to be displaced in newborns or younger children.

As evidenced in the described FIGS. 1 and 4, the present invention diaper provides as advantages the fact of being comfortably worn by a great number of users, of different heights and physical constitutions. It is rapidly placed, as well as extremely safe against loosening or displacements, thus preventing body fluids leakage, aspects which make the use thereof extremely desirable. The easy placement in a user is shown by a better adjustment in the region between the legs and on the waist in relation to state-of-the-art diapers, especially in newborns, and since the diaper is not a closed one, which would force introducing the child's legs in the orifices therefor, which is of great commodity and practical use thereof.

Having now described a preferred embodiment, it should be understood that the scope of the present invention encloses other possible variations, such as diapers having a differently shaped from the one shown in the figure, being limited only by the following claims content, including possible equivalents.

1. Diaper comprising a body fluid permeable material layer adapted to contact a user's body in use, a body fluid impermeable material layer, an absorbent core disposed between and enclosed by said respective fluid permeable and impermeable material layers, the diaper further including a pair of opposite side margins, a rear margin and a distal end, the distal end being opposite the rear margin and defining therebetween a longitudinal length, the rear margin being adjacent to both said opposite side margins, the rear margin and the distal end each having a respective transverse width, the rear margin comprising a continuous and ring shaped element adapted to be worn around a waist region of the user's body in use, wherein the distal end is freely extending and moveable with respect to the rear margin and being adapted to be releaseably attached to at least a portion of the rear margin.

2. Diaper, according to claim 1, wherein at least a portion of the ring shaped element is formed by at least two superposed elastic portions joined by transverse joining lines.

3. Diaper, according to claim 1 wherein the absorbent core is longitudinally disposed along the length of the diaper and closer to the distal end than to the rear margin.

4. Diaper, according to claim 1 wherein the side margins converge towards to the distal end.

5. Diaper, according to claim 1 wherein a ratio of the transverse width of the rear margin, in a plan view, to the transverse width of the distal end opposed to said margin is a value greater than 2.

6. Diaper, according to claim 5, wherein a ratio of the width of rear margin, in a plan view, to the transverse width of the distal end opposed to said margin is preferably equal to 3.

7. Diaper, according to claim 1 wherein the ratio of the longitudinal length of the diaper to the transverse width of the rear margin, in a plan view, is a value between 0.6 and 1.3.

8. Diaper, according to claim 7 wherein the ratio of the longitudinal length of the diaper to the transverse width of the rear margin, in a plan view, is preferably equal to 1.0.

9. A diaper according to claim 1 wherein the distal end further comprises an attachment element adapted to releasably attach the distal end to at least a portion of the rear margin.

10. A diaper according to claim 9 wherein the attachment element comprises a hook and loop fastener.
